(19) United States
(12) Patent Application Publication
(10) Pub. No.: US 2008/0280904 A1
(43) Pub. Date: Nov. 13, 2008

(54) N-Substituted Pyridinone or Pyrimidinone Compounds Useful as Soluble Epoxide Hydrolase Inhibitors

(76) Inventors: Anne Bettina Eldrup, Danbury, CT (US); Fariba Soleymanzadeh, Danbury, CT (US); Steven John Taylor, Southbury, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(21) Appl. No.: 12/089,136

(22) PCT Filed: Oct. 4, 2006
(86) PCT No.: PCT/US2006/039055

§ 371 Date: Apr. 3, 2008

Related U.S. Application Data
(60) Provisional application No. 60/724,574, filed on Oct. 7, 2005.

(51) Int. Cl. A61K 31/5377; C07D 213/64; A61K 31/4412; C07D 401/06; A61P 3/10; A61K 31/444; C07D 413/06

(52) U.S. Cl. 514/235.5; 546/291; 514/346; 544/131; 546/261; 514/335

(57) ABSTRACT

Disclosed are compounds active against soluble epoxide hydrolase (sEH), compositions thereof and methods of using and making same.

### CROSS REFERENCED RELATED APPLICATIONS

This Application is a National Phase 371 of PCT/US06/039055, filed Oct. 4, 2006, which is a PCT international filing of U.S. provisional application 60/724,574, filed Oct. 7, 2005.

### BACKGROUND OF THE INVENTION

1. Technical Field

This invention relates to compounds possessing anti-sEH activity and methods of using soluble epoxide hydrolase (sEH) inhibitors for diseases related to cardiovascular disease.

2. Background Information

Epoxide hydrolases are a group of enzymes ubiquitous in nature, detected in species ranging from plants to mammals. These enzymes are functionally related in that they all catalyze the addition of water to an epoxide, resulting in a diol. Epoxide hydrolases are important metabolizing enzymes in living systems and their diol products are frequently found as intermediates in the metabolic pathway of xenobiotics. Epoxide hydrolases are therefore important enzymes for the detoxification of epoxides by conversion to their corresponding, non-reactive diols.

In mammals, several types of epoxide hydrolases have been characterized including soluble epoxide hydrolase (sEH), also referred to as cytosolic epoxide hydrolase, cholesterol epoxide hydrolase, LTA₄ hydrolase, hepoxilin hydrolase, and microsomal epoxide hydrolase (Fretland and Omiecinski, Chemico-Biological Interactions, 129: 41-59 (2000)). Epoxide hydrolases have been found in all tissues examined in vertebrates including heart, kidney and liver (Vogel, et al., Eur J. Biochemistry, 126: 425-431 (1982); Schladt et al., Biochem. Pharmacol., 35: 3309-3316 (1986)). Epoxide hydrolases have also been detected in human blood components including lymphocytes (e.g. T-lymphocytes), monocytes, erythrocytes, platelets and plasma. In the blood, most of the sEH detected was present in lymphocytes (Seidegard et al., Cancer Research, 44: 3654-3660 (1984)).

The epoxide hydrolases differ in their specificity towards epoxide substrates. For example, sEH is selective for aliphatic epoxides such as epoxide fatty acids while microsomal epoxide hydrolase (mEH) is more selective for cyclic and arene epoxides. The primary known physiological substrates of sEH are four regioisomeric cis epoxides of arachidonic acid, 5,6-, 8,9-, 11,12-, and 14,15-epoxyeicosatrienoic acid, also known as epoxyeicosatrienoic acids or EETs. Also known to be substrates for sEH are epoxides of linoleic acid known as leukotoxin or isoleukotoxin. Both the EETs and the leukotoxins are generated by members of the cytochrome P450 monooxygenase family (Capdevila, et al., J. Lipid Res., 41: 163-181 (2000)).

EETs function as chemical autocrine and paracrine mediators in the cardiovascular and renal systems (Spector, et al, Progress in Lipid Research, 43: 55-90 (2004); Newman, et al., Progress in Lipid Research 44: 1-51 (2005)). EETs appear to be able to function as endothelial derived hyperpolarizing factor (EDHF) in various vascular beds due to their ability to cause hyperpolarization of the membranes of vascular smooth muscle cells with resultant vasodilation (Weintraub, et al., Circ. Res., 81: 258-267 (1997)). EDHF is synthesized from arachidonic acid by various cytochrome P450 enzymes in endothelial cells proximal to vascular smooth muscle (Quilley, et al., Brit. Pharm., 54: 1059 (1997); Quilley and McGiff, TIPS, 21: 121-124 (2000)); Fleming and Busse, Nephrol. Dial. Transplant, 13: 2721-2723 (1998)). In the vascular smooth muscle cells EETs provoke signaling pathways which lead to activation of BK_{Ca2+} channels (big Ca²⁺ activated potassium channels) and inhibition of L-type Ca²⁺ channels, ultimately resulting in hyperpolarization of membrane potential, inhibition of Ca²⁺ influx and relaxation (Li et al., Circ. Res., 85: 349-356 (1999)). Endothelium dependent vasodilation has been shown to be impaired in different forms of experimental hypertension as well as in human hypertension (Lind, et al., Blood Pressure, 9: 4-15 (2000)). Impaired endothelium dependent vasorelaxation is also a characteristic feature of the syndrome known as endothelial dysfunction (Goligorsky, et. al., Hypertension, 37[part 2]:744-748 (2001)). Endothelial dysfunction plays a significant role in a large number of pathological conditions including type 1 and type 2 diabetes, insulin resistance syndrome, hypertension, atherosclerosis, coronary artery disease, angina, ischemia, ischemic stroke, Raynaud's disease and renal disease. Hence, it is likely that enhancement of EETs concentration would have a beneficial therapeutic effect in patients where endothelial dysfunction plays a causative role. Other effects of EETs that may influence hypertension involve effects on kidney function. Levels of various EETs and their hydrolysis products, the DHETs, increase significantly both in the kidneys of spontaneously hypertensive rats (SHR) (Yu, et al., Circ. Res. 87: 992-998 (2000)) and in women suffering from pregnancy induced hypertension (Catella, et al., Proc. Natl. Acad. Sci. U.S.A., 87: 5893-5897 (1990)). In angiotensin II infused rats the treatment with a selective sEH inhibitor attenuated the afferent arteriolar diameter in the kidney and lowered urinary albumin secretion, a marker of compromised renal function, suggesting antihypertensive and renal vascular protective effects of increased EETs levels (Zhao, et al, 15: 1244-1253 (2004)). In the spontaneously hypertensive rat model, both cytochrome P450 and sEH activities were found to increase (Yu et al., Molecular Pharmacology, 57: 1011-1020 (2000)). Addition of a known sEH inhibitor was shown to decrease the blood pressure to normal levels. Furthermore, administration of a selective sEH inhibitor to angiotensin II treated rats was demonstrated to lower systolic blood pressure (Imig, et al, Hypertension, 39: 690-694 (2002)). Finally, male soluble epoxide hydrolase null mice exhibited a phenotype characterized by lower blood pressure than their wild-type counterparts (Sinal, et al., J. Biol. Chem., 275: 40504-40510 (2000)).

EETs, especially 11,12-EET, also have been shown to exhibit anti-inflammatory properties (Node, et al., Science, 285: 1276-1279 (1999); Campbell, TIPS, 21: 125-127 (2000); Zeldin and Liao, TIPS, 21: 127-128 (2000)). Node, et al. have demonstrated 11,12-EET decreases expression of cytokine induced endothelial cell adhesion molecules, especially VCAM-1. They further showed that EETs prevent leukocyte adhesion to the vascular wall and that the mechanism responsible involves inhibition of NF-κB and IκB kinase. Vascular inflammation plays a role in endothelial dysfunction (Kessler, et al., Circulation, 99: 1878-1884 (1999)). Hence, the ability of EETs to inhibit the NF-κB pathway should also help ameliorate this condition. In addition, the administration of EETs and/or the administration of a selective sEH inhibitor was demonstrated to attenuate tobacco smoke induced inflammation, as assessed total bronchoalveolar lavage cell numbers and concomittant reduction in neutrophils, alveolar macrophages, and lymphocytes (Smith, et al, 102: 2186-2191 (2005)).

In addition to the physiological effect of some substrates of sEH (EETs, mentioned above), some diols, i.e. DHETs, produced by sEH may have potent biological effects. For example, sEH metabolism of epoxides produced from linoleic acid (leukotoxin and isoleukotoxin) produces leukotoxin and isoleukotoxin diols (Greene, et al., Arch. Biochem. Biophys. 376(2): 420-432 (2000)). These diols were shown to be toxic to cultured rat alveolar epithelial cells, increasing intracellular calcium levels, increasing intercellular junction permeability and promoting loss of epithelial integrity (Moghaddam et al., Nature Medicine, 3: 562-566 (1997)). Therefore these diols could contribute to the etiology of diseases such as adult respiratory distress syndrome where lung leukotoxin levels have been shown to be elevated (Ishizaki, et al., Pulm. Pharm. & Therap., 12: 145-155 (1999)). Hammock, et al. have disclosed the treatment of inflammatory diseases, in particular adult respiratory distress syndrome and other acute inflammatory conditions mediated by lipid metabolites, by the administration of inhibitors of epoxide hydrolase (WO 98/06261; U.S. Pat. No. 5,955,496).

A number of classes of sEH inhibitors have been identified. Among these are chalcone oxide derivatives (Miyamoto, et al. Arch. Biochem. Biophys., 254: 203-213 (1987)) and various trans-3-phenylglycidols (Dietze, et al., Biochem. Pharm. 42: 1163-1175 (1991); Dietze, et al., Comp. Biochem. Physiol. B, 104: 309-314 (1993)).

More recently, Hammock et al. have disclosed certain biologically stable inhibitors of sEH for the treatment of inflammatory diseases, for use in affinity separations of epoxide hydrolases and in agricultural applications (U.S. Pat. No. 6,150,415). The Hammock '415 patent also generally describes that the disclosed pharmacophores can be used to deliver a reactive functionality to the catalytic site, e.g., alkylating agents or Michael acceptors, and that these reactive functionalities can be used to deliver fluorescent or affinity labels to the enzyme active site for enzyme detection (col. 4, line 66 to col. 5, line 5). Certain urea and carbamate inhibitors of sEH have also been described in the literature (Morisseau et al., Proc. Natl. Acad. Sci., 96: 8849-8854 (1999); Argiriadi et al., J. Biol. Chem., 275 (20): 15265-15270 (2000); Nakagawa et al. Bioorg. Med. Chem., 8: 2663-2673 (2000); US 2005/0026844 and Kim, et al., J. Med. Chem. 47(8): 2110-2122 (2004) both of which describe inhibitors with additional, tethered oxo pharmacophores).

WO 00/23060 discloses a method of treating immunological disorders mediated by T-lymphocytes by administration of an inhibitor of sEH. Several 1-(4-aminophenyl)pyrazoles are given as examples of inhibitors of sEH.

U.S. Pat. No. 6,150,415 to Hammock is directed to a method of inhibiting an epoxide hydrolase, using compounds having the structure

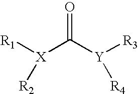

wherein X and Y is each independently nitrogen, oxygen, or sulfur, and X can further be carbon, at least one of R1-R4 is hydrogen, R2 is hydrogen when X is nitrogen but is not present when X is sulfur or oxygen, R4 is hydrogen when Y is nitrogen but is not present when Y is sulfur or oxygen, R1 and R3 is each independently H, C1-20 substituted or unsubstituted alkyl, cycloalkyl, aryl, acyl, or heterocyclic. Related to the Hammock patent is U.S. Pat. No. 6,531,506 to Kroetz et al. which claims a method of treating hypertension using of an inhibitor of epoxide hydrolase, also claimed are methods of treating hypertension using compounds similar to those described in the Hammock patent. Neither of these patents teaches or suggests methods of treating cardiovascular diseases using the particular sEH inhibitors described herein.

As outlined in the discussion above, inhibitors of sEH are useful therefore, in the treatment of cardiovascular diseases such as endothelial dysfunction either by preventing the degradation of sEH substrates that have beneficial effects or by preventing the formation of metabolites that have adverse effects.

All references cited above and throughout this application are incorporated herein by reference in their entirety.

### BRIEF SUMMARY OF THE INVENTION

It is therefore an object of the invention to provide compounds active as sEH inhibitors of the formula I as described herein below.

It is a further object of the invention to provide a method of treating hypertension by administering to a patient a compound of the formula I as described herein below. It is yet a further object to provide methods of making the compounds described herein below.

### DETAILED DESCRIPTION OF THE INVENTION

In one generic aspect of the invention, there is provided a compound of the formula (I):

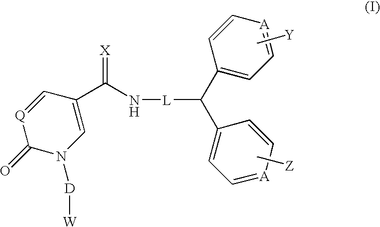

Each A is independently nitrogen or C—H such that each of the ring of which A is a member may be pyridinyl or phenyl, said pyridinyl or phenyl are optionally substituted by Y or Z;
Y and Z on their respective rings are in the meta or para position, and are independently F, Cl, Br, CN, OR, R, —S(O)₂R, —C(O)NRR or —S(O)₂NRR, wherein R is independently hydrogen or C1-5 alkyl unsubstituted or substituted with hydroxy, amino, C₁₋₄ alkoxy, C₁₋₄ alkylamino, C₁₋₄ alkylthio, or one to three fluorine atoms;
L is an ethylene linker optionally substituted with hydroxy, amino, C₁₋₄ alkoxy C₁₋₄ alkylamino, C₁₋₄ alkylthio, or one to three fluorine atoms;

### X is O or S;

### Q is N or CH;

D is a bond, or a methylene or ethylene linker, wherein a —CH₂— group of said linker may be replaced by —C(O)—;
W is hydrogen, C1-5 alkyl, cyano, carbocycle, heterocylyl, aryloxy, C1-4 alkyloxy, OH or heteroaryl; each being unsubstituted or substituted with hydroxy, amino, halogen, cyano, carboxy, carboxamido, C1-4 alkyl, C1-4 alkylthio, C1-4 alkyloxy, C1-4 alkylamino, C1-4 dialkylamino, C3-6 cycloalkylamino, di(C3-6 cycloalkyl)amino, C1-4 alkylsulfonyl, C1-4 alkyloxycarbonyl or C1-4 alkylamidocarbonyl;
wherein if D is a bond then W is cyano,
or the pharmaceutically acceptable salts thereof.

In another embodiment there is provided a compound according to the embodiment immediately above and wherein:

### Q is CH;

### X is O;

Y and Z, if present, on their respective rings are in the meta or para position, and are independently F, Cl, —S(O)₂R or —C(O)NRR wherein R is independently hydrogen or C1-5 alkyl;
W is hydrogen, cyano, C3-6 cycloalkyl, aryl, phenoxy, C1-4 alkyloxy, OH, C1-5 alkyl, heterocylyl chosen from piperidinyl, morpholinyl, thiomorpholinyl, piperazinyl, pyrrolidinyl and dioxolanyl or heteroaryl chosen from pyrazolyl, pyrrolyl, imidazolyl, furanyl, pyranyl, thienyl, thiazolyl, oxazolyl, pyridinyl, pyrimidinyl, pyrazinyl, pyridazinyl and

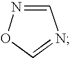

each being unsubstituted or substituted with hydroxy, amino, halogen, cyano, carboxy, carboxamido, C1-4 alkyl, C1-4 alkylthio, C1-4 alkyloxy, C1-4 alkylamino, C1-4 dialkylamino, C3-6 cycloalkylamino, di(C3-6 cycloalkyl)amino, C1-4 alkylsulfonyl, C1-4 alkyloxycarbonyl or C1-4 alkylamidocarbonyl.

In another embodiment there is provided a compound according to the embodiment immediately above and wherein:

L is an ethylene linker;
W is hydrogen, cyano, piperidinyl, morpholinyl, phenyl, phenoxy, C₁₋₄ alkyloxy, OH, C1-5 alkyl, pyridinyl or

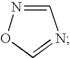

each being unsubstituted or substituted with hydroxy or C₁₋₄ alkyloxy.

In another generic aspect of the invention, there is provided a compound of the formula (II):

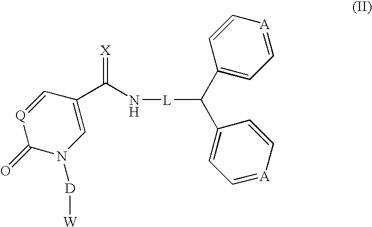

wherein for the Formula (II), the component

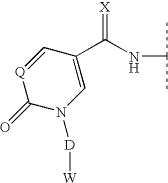

is chosen from A1-A14 in the table I below; in combination with any component

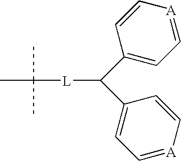

chosen from B1-B11 in the table I below;

| | 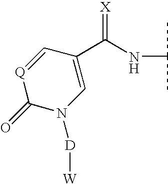 |
|---|---|
| A1 | 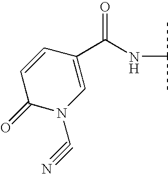 |
| A2 | 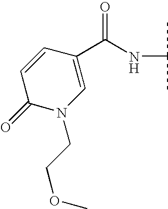 |
| A3 | 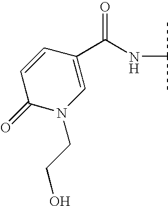 |
| A4 | 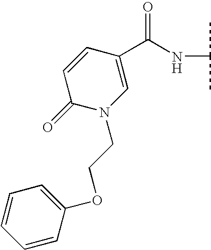 |
| A5 | 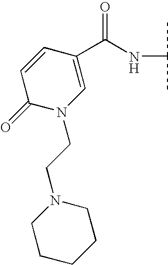 |
| A6 | 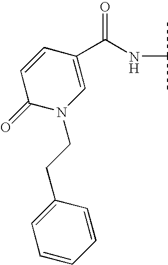 |
| A7 | 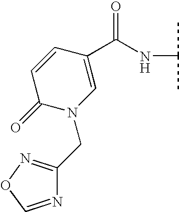 |
| A8 | 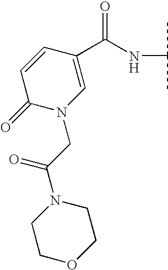 |
| A9 | 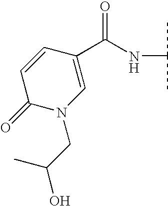 |
| A10 | 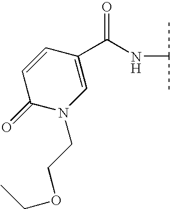 |
| A11 | 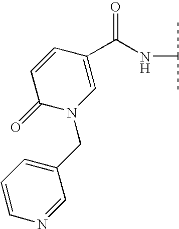 |
| A12 | 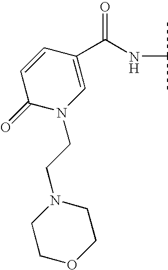 |
| A13 | 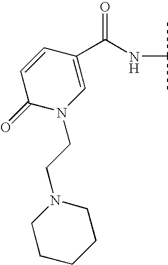 |
| A14 | 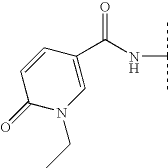 |
| | 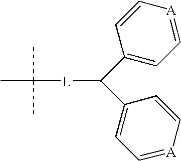 |
| B1 | 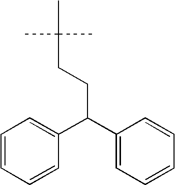 |
| B2 | 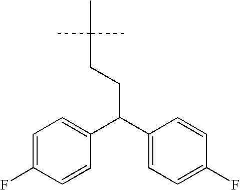 |
| B3 | 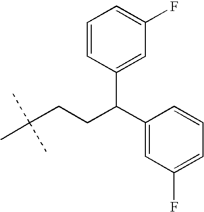 |
| B4 | 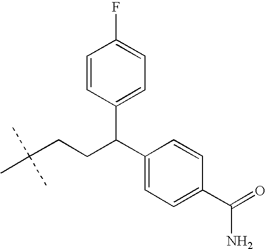 |
| B5 | 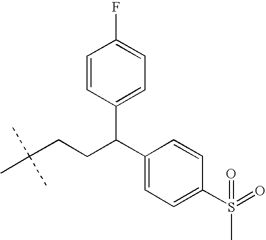 |
| B6 | 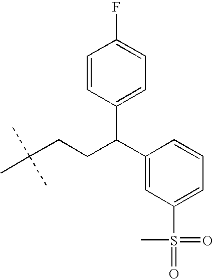 |
| B7 | 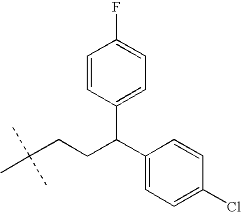 |
| B8 | 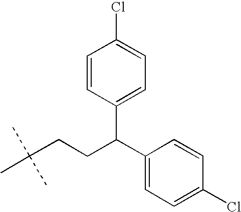 |
| B9 | 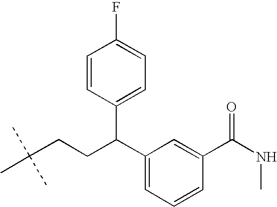 |
| B10 | 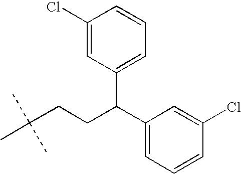 |
| B11 | 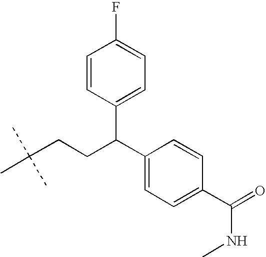 |

or the pharmaceutically acceptable salts thereof.

In another embodiment of the invention there is provided the following compounds which can be made according to the general synthetic procedures and examples which follow:

TABLE II

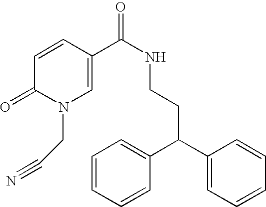

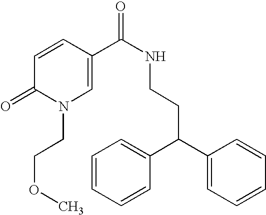

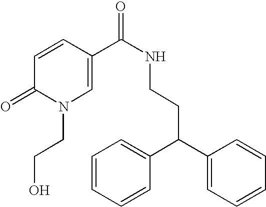

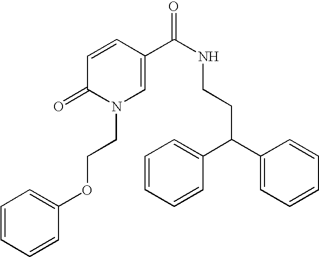

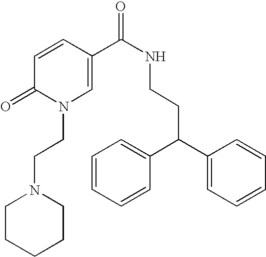

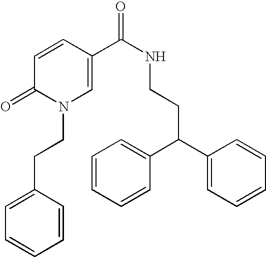

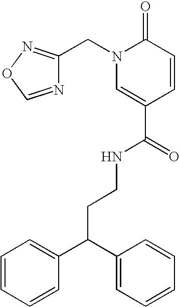

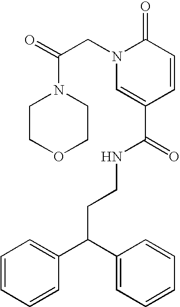

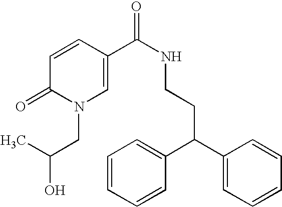

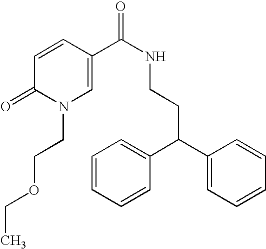

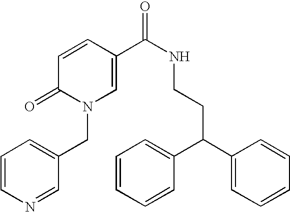

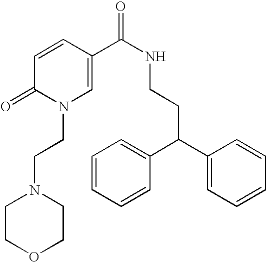

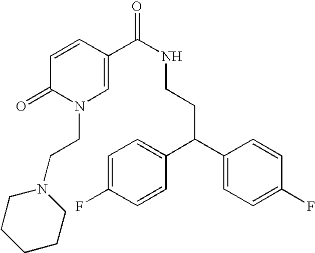

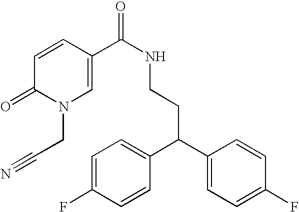

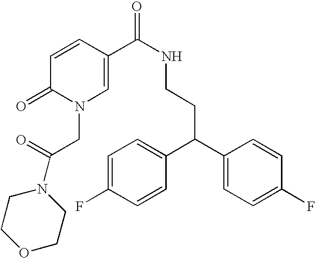

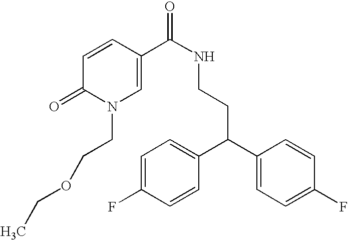

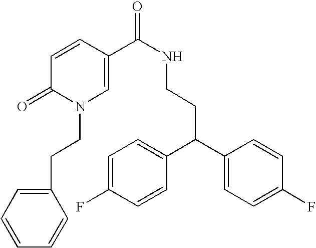

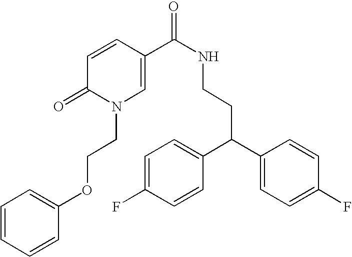

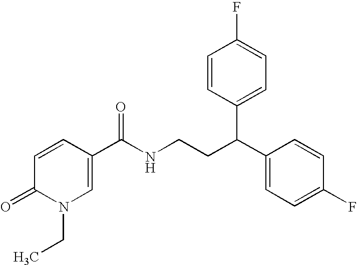

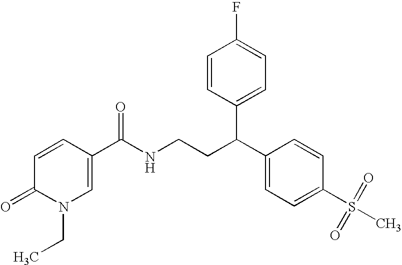

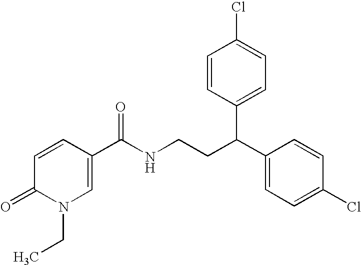

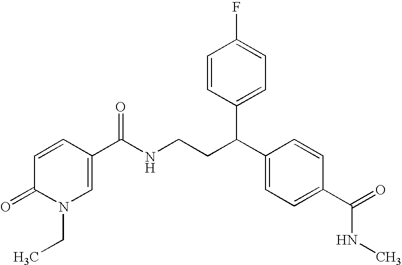

or the pharmaceutically acceptable salts thereof.

In all the compounds disclosed hereinabove in this application, in the event the nomenclature is in conflict with the structure, it shall be understood that the compound is defined by the structure.

The invention includes the use of any compounds of described above containing one or more asymmetric carbon atoms may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Some of the compounds of the invention can exist in more than one tautomeric form. The invention includes methods using all such tautomers.

All terms as used herein in this specification, unless otherwise stated, shall be understood in their ordinary meaning as known in the art. For example, C₁₋₄alkoxy includes the organic radical C₁₋₄alkyl with a terminal oxygen, such as methoxy, ethoxy, propoxy, butoxy.

All organic radicals: alkyl, alkenyl and alkynyl groups, or such groups which are incorporated in other radicals such as acyl and alkoxy, shall be understood as being branched or unbranched where structurally possible and unless otherwise specified, and may be partially or fully halogenated.

The term “lower” referred to above and hereinafter in connection with organic radicals or compounds respectively defines such as branched or unbranched with up to and including 7, preferably up to and including 4 and advantageously one or two carbon atoms.

A cyclic group shall be understood to mean carbocycle, heterocycle or heteroaryl, each may be partially or fully halogenated.

An acyl group is a radical defined as —C(O)—R, where R is an organic radical or a cyclic group. Acyl represents, for example, carbocyclic or heterocyclic aroyl, cycloalkylcarbonyl, (oxa or thia)-cycloalkylcarbonyl, lower alkanoyl, (lower alkoxy, hydroxy or acyloxy)-lower alkanoyl, (mono- or di-carbocyclic or heterocyclic)-(lower alkanoyl or lower alkoxy-, hydroxy- or acyloxy-substituted lower alkanoyl), or biaroyl.

Carbocycles include hydrocarbon rings containing from three to fourteen carbon atoms. These carbocycles may be either aromatic either aromatic or non-aromatic ring systems. The non-aromatic ring systems may be mono- or polyunsaturated, monocyclic, bicyclic or tricyclic and may be bridged. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, benzyl, indanyl, indenyl, benzocyclobutanyl, dihydronaphthyl, tetrahydronaphthyl, naphthyl, decahydronaphthyl, benzocycloheptanyl, adamantyl, norbornyl, fluorene, and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure. Unless otherwise stated, heterocycles include but are not limited to, for example pyrrolidinyl, pyrrolinyl, morpholinyl, thiomorpholinyl, thiomorpholinyl sulfoxide, thiomorpholinyl sulfone, dioxalanyl, piperidinyl, piperazinyl, tetrahydrofuranyl, tetrahydropyranyl, tetrahydrofuranyl, 1,3-dioxolanone, 1,3-dioxanone, 1,4-dioxanyl, piperidinonyl, tetrahydropyrimidonyl, pentamethylene sulfide, pentamethylene sulfoxide, pentamethylene sulfone, tetramethylene sulfide, tetramethylene sulfoxide and tetramethylene sulfone.

The term “heteroaryl” shall be understood to mean an aromatic 5-8 membered monocyclic or 8-11 membered bicyclic ring containing 1-4 heteroatoms such as N, O and S. Unless otherwise stated, such heteroaryls include aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazolo[3,4-b]pyridinyl, tubercidinyl, oxazo[4,5-b]pyridinyl, imidazo[4,5-b]pyridinyl and

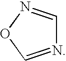

The term “heteroatom” as used herein shall be understood to mean atoms other than carbon such as oxygen, nitrogen, sulfur and phosphorous.

As used herein, “nitrogen” and “sulfur” include any oxidized form of nitrogen and sulfur and the quaternized form of any basic nitrogen. All heteroatoms in open chain or cyclic radicals include all oxidized forms.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative and/or is partially or fully halogenated. For example, quinolinyl may include decahydroquinolinyl and tetrahydroquinolinyl, naphthyl may include it's hydrogenated derivatives such as tetrahydranaphthyl. Other partially or fully hydrogenated derivatives of the aryl and heteroaryl compounds described herein will be apparent to one of ordinary skill in the art.

The term “halogen” as used in the present specification shall be understood to mean bromine, chlorine, fluorine or iodine, preferably fluorine. The definitions “partially or fully halogenated”; partially or fully fluorinated; “substituted by one or more halogen atoms”, includes for example, mono, di or tri halo derivatives on one or more carbon atoms. For alkyl, a nonlimiting example would be —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of the invention. A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the invention.

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the invention. Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

The compounds described herein are either commercially available or can be made by methods and any necessary intermediates well known in the art.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the scheme below are either commercially available or easily prepared from commercially available materials by those skilled in the art.

### General Synthetic Methods

The invention also provides processes for making compounds of Formula (I) and (II). In all schemes, unless specified otherwise, A, D, L, Q, W, Y and Z in the formulas below shall have the meaning of A, D, L, Q, W, Y and Z in Formula (I) and (II) of the invention described herein above.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC), if desired, and intermediates and products may be purified by chromatography on silica gel and/or by recrystallization. The appropriately substituted starting materials and intermediates used in the preparation of compounds of the invention are either commercially available or readily prepared by methods known in the literature to those skilled in the art, and are illustrated in the synthetic examples below. Reference in this regard can be made to U.S. provisional application Nos. 60/678,828 and 60/678,871, incorporated herein be reference.

Compounds of Formula (I) and (II) may be synthesized by the method illustrated in Scheme 1

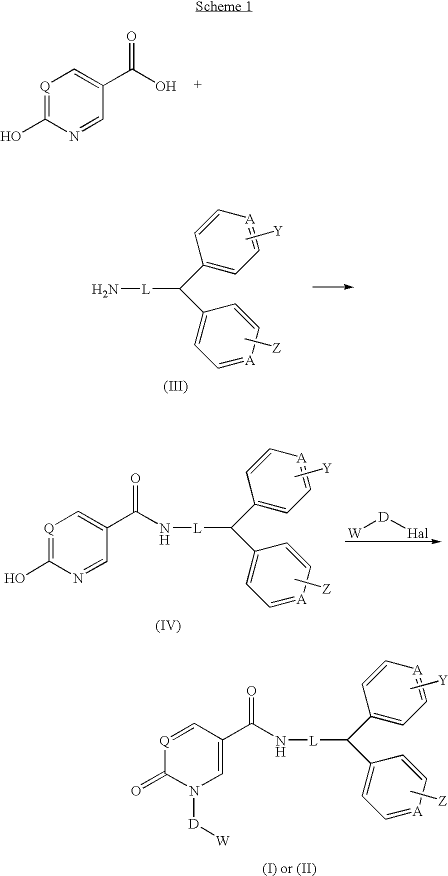

Amide coupling of the carboxylic acid with the desired amine (III) provides the compound of formula (IV). Standard peptide coupling reactions known in the art (see for example M. Bodanszky, 1984, The Practice of Peptide Synthesis, Springer-Verlag) may be employed in these syntheses. An example of suitable coupling conditions is treatment of a solution of the carboxylic acid in a suitable solvent such as DMF with EDC, HOBT, and a base such as diisopropylethylamine, followed by the desired amine.

Alternatively, reaction of the carboxylic acid with reagents such as oxalyl chloride provides the corresponding acid chloride. Reaction of the acid chloride with the desired amine (III) in a suitable solvent provides the compound of formula (IV). N-alkylation of the compound of formula (IV) with suitable alkylating agent such as W-D-Hal, wherein Hal is Cl, Br or I, and optionally potassium iodide, in a suitable solvent, in the presence of a suitable base provides the desired product of formula (I) or (II). Further modification of the initial product of formula (I) or (II) by methods known in the art and illustrated in the Examples below, may be used to prepare additional compounds of this invention.

Compounds of Formula (I) and (II) may also be synthesized by the method illustrated in Scheme 2

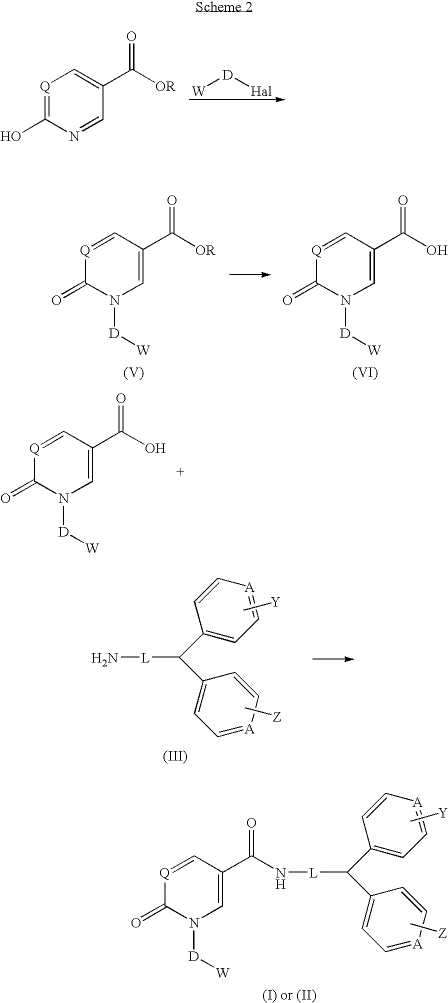

N-alkylation of the starting hydroxyl pyridine ester with a suitable alkylating agent such as W-D-Hal, wherein Hal is Cl, Br or I, and optionally potassium iodide, in a suitable solvent, in the presence of a suitable base, provides a compound of formula (V). Hydrolysis of the ester of formula (V) with a suitable base, such as lithium hydroxide, in a suitable solvent provides N-alkylated nicotinic acid of formula (VI).

Amide coupling of the carboxylic acid with the desired amine (III) provides the compound of formula (I) or (II) as in scheme 1. Alternatively, reaction of the carboxylic acid with reagents such as oxalyl chloride provides the corresponding acid chloride. Reaction of the acid chloride with the desired amine (III) in a suitable solvent provides the compound of formula (I) or (II)

Intermediate amine of formula (III) may be prepared by the method outlined in Scheme 3

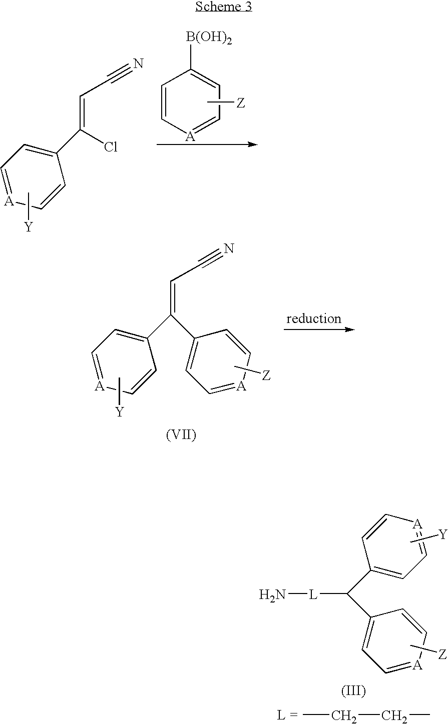

Reaction of an appropriately substituted 3-chloro acrylonitrile with an appropriately substituted aryl/heteroaryl boronic acid, in the presence of a suitable coupling catalyst, in a suitable solvent, provides the coupled product (VII). Reduction of the coupled product (VII) with a suitable reducing agent, provides the desired amine (III)

The appropriately substituted starting materials and intermediates used in the preparation of compounds of the invention are either commercially available or readily prepared by methods known in the literature to those skilled in the art, and are illustrated in the synthetic examples below.

### EXAMPLE 1

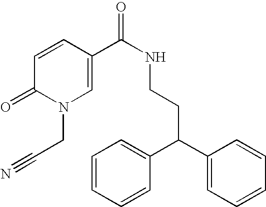

### 1-Cyanomethyl-6-oxo-1,6-dihydro-pyridine-3-carboxylic acid (3,3-diphenyl-propyl)-amide

### Step A

### N-(3,3-diphenyl-propyl)-6-hydroxy-nicotinamide

To a solution of 6-hydroxy amino-nicotinic acid (0.065 g, 0.473 mmol) in N,N,dimethylformamide (4 mL) is added 3,3-diphenyl-propylamine (0.100 g, 0.473 mmol) followed by the addition of 1-hydroxybenzotriazole (0.127 g, 0.946 mmol), 1-ethyl-3-(3-dimethylaminopropyl)-carbodiimide hydrochloride (0.180 g, 0.946 mmol) and diisopropylethylamine (0.247 mL, 1.419 mmol). The reaction is stirred overnight. The mixture is diluted with water and the product is extracted using dichloromethane. The organic extract is passed through a cartridge containing silica gel and magnesium sulfate. The resulting compound is purified by flash chromatography using methanol/dichloromethane (2:98 through 10:90) as the eluent and evaporated in vacuo to provide the desired compound (0.041 g, 26.3%).

### Step B

### 1-Cyanomethyl-6-oxo-1,6-dihydro-pyridine-3-carboxylic acid (3,3-diphenyl-propyl)-amide

To a solution of the product from Step A (0.050 g, 0.150 mmol) in acetonitrile (5 mL) is added bromoacetonitrile (0.018 g, 0.150 mmol) followed by the addition of PS-TBD (0.383 g, 0.450 mmol). The mixture is stirred overnight, filtered, and evaporated in vacuo to give the desired product (0.011 g, 19.7%). LCMS: 372.33 (M+H⁺).

### EXAMPLE 2

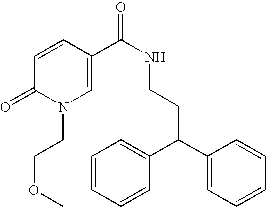

### 1-(2-Methoxy-ethyl)-6-oxo-1,6-dihydro-pyridine-3-carboxylic acid (3,3-diphenyl-propyl)-amide

The compound is prepared using the procedure from Example 1, starting from the product of Example 1, Step A (0.050 g, 0.150 mmol) and 1-bromo-2-methoxy-ethane (0.032 g, 0.225 mmol), and purified by preparative TLC using methanol/dichloromethane as the eluent to give the desired product (0.010 g, 17.1%). LCMS: 391.48 (M+H⁺).

### EXAMPLE 3

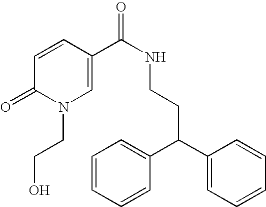

### 1-(2-Hydroxy-ethyl)-6-oxo-1,6-dihydro-pyridine-3-carboxylic acid (3,3-diphenyl-propyl)-amide

The compound is prepared using the procedure from Example 1, starting from the product of Example 1, Step A and 2-bromoethanol (0.037 g, 0.300 mmol), and purified by preparative TLC using methanol/dichloromethane as the eluent to give the desired product (0.028 g, 24.8%). LCMS: 377.38 (M+H⁺).

### EXAMPLE 4

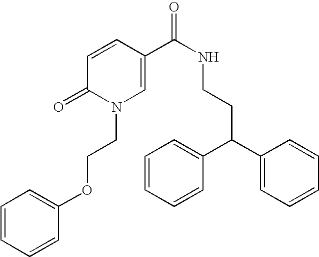

### 6-Oxo-1-(2-phenoxy-ethyl)-1,6-dihydro-pyridine-3-carboxylic acid (3,3-diphenyl-propyl)-amide

The compound is prepared using the procedure from Example 1, starting from the product of Example 1, Step A and 2-bromoethoxy benzene (0.054 g, 0.270 mmol). The product is purified by recrystallization from hexane/dichloromethane to give the desired product (0.015 g, 12.4%). LCMS: 453.37 (M+H⁺).

### EXAMPLE 5

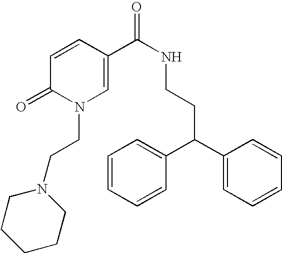

### 6-Oxo-1-(2-piperidin-1-yl-ethyl)-1,6-dihydro-pyridine-3-carboxylic acid (3,3-diphenylpropyl)-amide

The compound is prepared and purified using the procedure from Example 3, starting from the product of Example 1, Step A and 2-chloroethoxy piperidine (0.040 g, 0.270 mmol), to give the desired product (0.049 g, 41.6%). LCMS: 444.43 (M+H⁺).

### EXAMPLE 6

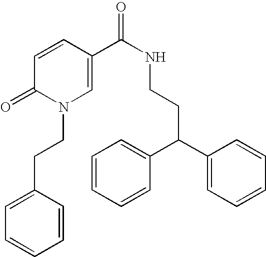

### 6-Oxo-1-phenethyl-1,6-dihydro-pyridine-3-carboxylic acid (3,3-diphenyl-propyl)-amide

The compound is prepared and purified using the procedure from Example 1, starting from the product of Example 1, Step A and 2-bromoethyl benzene (0.050 g, 0.271 mmol), to give the desired product (0.025 g, 21.1%). LCMS: 437.37 (M+H⁺).

### EXAMPLE 7

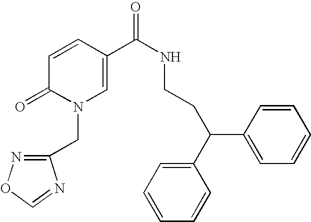

### 1-[1,2,4]Oxadiazol-3-yl-methyl-6-oxo-1,6-dihydro-pyridine-3-carboxylic acid (3,3-diphenyl-propyl)-amide

The compound is prepared and purified using the procedure from Example 1, starting from the product of Example 1, Step A and 3-chloromethyl-[1,2,4]oxadiazole (0.090 g, 0.270 mmol), to give the desired product (0.040 g, 35.7%). LCMS: 415.37 (M+H⁺).

### EXAMPLE 8

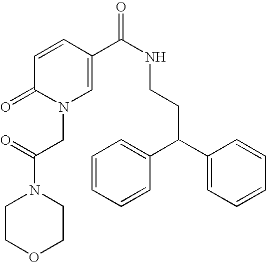

### 1-(2-Morpholin-4-yl-2-oxo-ethyl)-6-oxo-1,6-dihydro-pyridine-3-carboxylic acid (3,3-diphenyl-propyl)-amide

The compound is prepared using the procedure from Example 1, starting from the product of Example 1, Step A and 2-chloro-1-morpholine-4-yl-ethanol (0.090 g, 0.270 mmol) and purified by flash chromatography using methanol/dichloromethane (5:95), to give the desired product (0.041 g, 26.3%). LCMS: 460.39 (M+H⁺).

### EXAMPLE 9

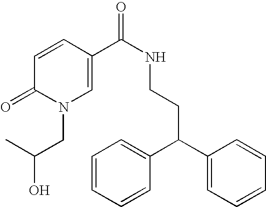

### 1-(2-Hydroxy-propyl)-6-oxo-1,6-dihydro-pyridine-3-carboxylic acid (3,3-diphenyl-propyl)-amide

The compound is prepared using the procedure from Example 1, starting from the product of Example 1, Step A and 1-bromo-propan-2-ol (0.042 g, 0.300 mmol), and purified by flash chromatography using methanol in dichloromethane (5:95) to give the desired product (0.008 g, 7.3%). LCMS: 391.36 (M+H⁺).

### EXAMPLE 10

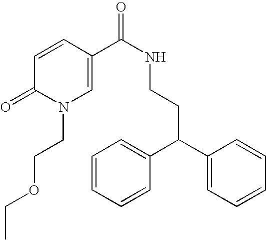

### 1-(2-Ethoxy-ethyl)-6-oxo-1,6-dihydro-pyridine-3-carboxylic acid (3,3-diphenyl-propyl)-amide

The compound is prepared using the procedure in Example 1, starting from the product of Example 1, Step A (0.100 g, 0.300 mmol) and 1-bromo-2-ethoxy-ethane (0.046 g, 0.300 mmol) and purified by preparative TLC using methanol/dichloromethane (5:95) as the eluent, to give the desired product (0.030 g, 24.7%). LCMS: 405.37 (M+H⁺).

### EXAMPLE 11

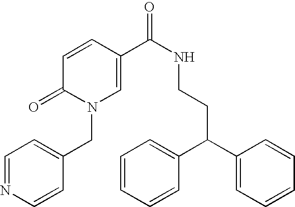

### 6-Oxo-1-pyridin-3-ylmethyl-1,6-dihydro-pyridine-3-carboxylic acid (3,3-diphenyl-propyl)-amide

The compound is prepared using the procedure from Example 1, starting from the product of Example 1, Step A (0.090 g, 0.270 mmol) and 3-bromomethyl-pyridine hydrobromide (0.068 g, 0.270 mmol) and purified by preparative TLC using methanol/dichloromethane (5:95) as the eluent, to give the desired product (0.015 g, 13.6%). LCMS: 424.34 (M+H⁺).

### EXAMPLE 12

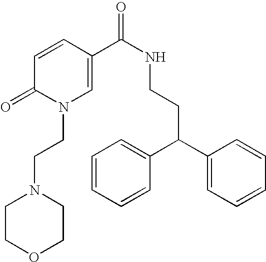

### 1-(2-Morpholin-4-yl-ethyl)-6-oxo-1,6-dihydro-pyridine-3-carboxylic acid (3,3-diphenylpropyl)-amide

The compound is prepared using the procedure in Example 1, starting from the product of Example 1, Step A (0.090 g, 0.270 mmol) and 4-(2-chloroethyl)-morpholine (0.041 g, 0.270 mmol) and purified by preparative TLC using methanol/dichloromethane (5:95) as the eluent, to give the desired product (0.007 g, 5.7%). LCMS: 446.38 (M+H⁺).

### EXAMPLE 13

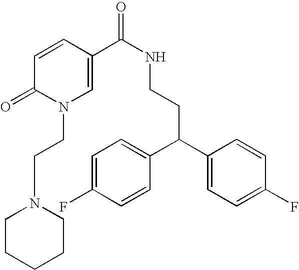

### 6-Oxo-1-(2-piperidin-1-yl-ethyl)-1,6-dihydro-pyridine-3-carboxylic acid [3,3-bis-(4-fluorophenyl)-propyl]-amide

### Step A

### 3,3-Bis-(4-fluoro-phenyl)-acrylonitrile

To a solution of cyanomethyl-phosphonic acid diethyl ester (14.6 g, 91.66 mmol) in acetonitrile (2.5 mL), is added washed and dried sodium hydride (60% dispersion in mineral oil) (4.399 g, 183.3 mmol) and stirred for a few minutes. Bis-(4-fluoro-phenyl)methanone (20.00 g, 91.66 mmol) is added to the mixture and it becomes dark red. The mixture is then put in smaller batches in microwave at 100° C. for 30 minutes. The reaction mixtures are evaporated in vacuo and triturated with diethylether. The ether phase is evaporated in vacuo to provide the desired product.

### Step B

### 3,3-Bis-(4-fluoro-phenyl)-propionitrile

To the product from Step A (0.315 g, 1.187 mmol) in ethanol is added palladium on carbon (10%, 0.100 g). The mixture is stirred under hydrogen overnight. The solution is filtered and the filtrate is evaporated in vacuo to provide the desired product.

### Step C

### 3,3-Bis-(4-fluoro-phenyl)-propylamine

To the solution of the product from Step B (0.336 g, 1.26 mmol) in dry tetrahydrofuran (4 mL) which is cooled down to 0° C., is added LiAlH₄ in tetrahydrofuran (1 M, 1.7 mL, 1.7 mmol) dropwise. The mixture is stirred at 0° C. for 1.5 hour and allowed to warm to room temperature and stirred for another 1.5 hour. Sodium bicarbonate is added to the reaction mixture, the mixture is passed through a layer of magnesium sulfate, and the filtrate is evaporated in vacuo to provide the desired product (17.00 g, 76.9%).

### Step D

### 6-Oxo-1,6-dihydro-pyridine-3-carboxylic acid [3,3-bis-(4-fluoro-phenyl propyl]-amide

This compound is prepared according to the procedure in Example 1, Step A, starting from the product of Step C (0.2 g, 0.809 mmol) and 6-hydroxy nicotinic acid (0.1 g, 0.719 mmol). The crude product is purified by preparative TLC using methanol/dichloromethane, (5:95) as the eluent to give the desired product (0.02 g, 7.6%).

### Step E

### 6-Oxo-1-(2-piperidin-1-yl-ethyl)-1,6-dihydro-pyridine-3-carboxylic acid [3,3-bis-(4-fluorophenyl)-propyl]-amide

This compound is prepared and purified using the procedure from Example 1, Step B, starting from the product of Step D (0.090 g, 0.270 mmol) and 1-chloro-2-ethoxy-piperidine (0.040 g, 0.270 mmol, and purified on preparative TLC using methanol/dichloromethane, (5:95) as the eluent to give the desired product (0.049 g, 41.6%). LCMS: 480.37 (M+H⁺).

### EXAMPLE 14

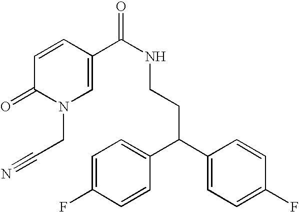

### 1-Cyanomethyl-6-oxo-1,6-dihydro-pyridine-3-carboxylic acid [3,3-bis-(4-fluoro-phenyl)-propyl]-amide

The compound is prepared and purified using the procedure from Example 1 starting from the product of Example 13, Step D (0.050 g, 0.136 mmol) and bromoacetonitrile (0.016 g, 0.136 mmol), to give the desired product (0.048 g, 87.5%). LCMS: 408.33 (M+H⁺).

### EXAMPLE 15

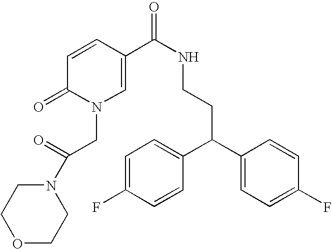

### 1-(2-Morpholin-4-yl-2-oxo-ethyl)-6-oxo-1,6-dihydro-pyridine-3-carboxylic acid [3,3-bis(4-fluoro-phenyl)-propyl]-amide

The compound is prepared using the procedure from Example 1, starting from the product of Example 13, Step D (0.050 g, 0.136 mmol) and 2-chloro-1-morpholin-4-yl-ethanone (0.022 g, 0.136 mmol), to give the desired product (0.031 g, 46.4%). LCMS: 496.35 (M+H⁺).

### EXAMPLE 16

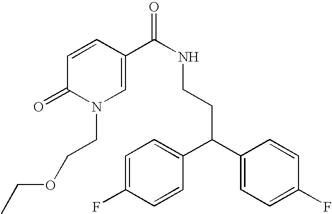

### 1-(2-Ethoxy-ethyl)-6-oxo-1,6-dihydro-pyridine-3-carboxylic acid [3,3-bis-(4-fluorophenyl)-propyl]-amide

The compound is prepared using the procedure from Example 1, starting from the product of Example 13, Step D (0.050 g, 0.136 mmol) and 1-bromo-2-ethoxy-ethane (0.021 g, 0.136 mmol), to give the desired product (0.021 g, 35.1%). LCMS: 441.36 (M+H⁺).

### EXAMPLE 17

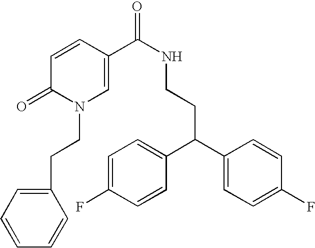

### 6-Oxo-1-phenethyl-1,6-dihydro-pyridine-3-carboxylic acid [3,3-bis-(4-fluoro-phenyl)-propyl]-amide

The compound is prepared using the procedure from Example 1, starting from the product of Example 13, Step D (0.050 g, 0.136 mmol) and (2-bromo-ethyl)-benzene (0.025 g, 0.136 mmol) and recrystallized over hexane to give the desired product (0.027 g, 42.5%). LCMS: 473.35 (M+H⁺).

### EXAMPLE 18

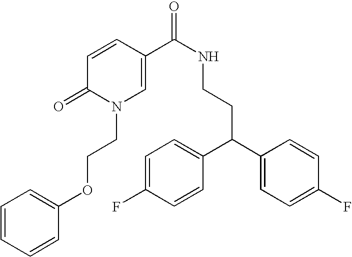

### 6-Oxo-1-(2-phenoxy-ethyl)-1,6-dihydro-pyridine-3-carboxylic acid [3,3-bis-(4-fluorophenyl)-propyl]-amide

The compound is prepared using the procedure from Example 1 starting from the product of Example 13, Step D (0.050 g, 0.136 mmol) and (2-bromo-ethoxy)-benzene (0.027 g, 0.136 mmol), and purified on preparative TLC using methanol/dichloromethane (5:95) as the eluent to give the desired product (0.023 g, 34.9%). LCMS: 489.35 (M+H⁺).

### EXAMPLE 19

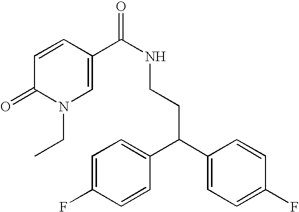

### 1-Ethyl-6-oxo-1,6-dihydro-pyridine-3-carboxylic acid [3,3-bis-(4-fluoro-phenyl)-propyl]-amide

The compound is prepared using the procedure from Example 1, starting from the product of Example 13, Step D (0.1 g, 0.271 mmol) and bromoethane (0.048, 0.440 mmol) and purified by flash chromatography using methanol/dichloromethane (5:95) as the eluent, to give the desired product (0.048 g, 44.6%). LCMS: 397.435 (M+H⁺).

### EXAMPLE 20

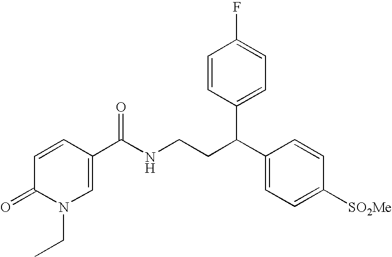

### 1-Ethyl-6-oxo-1,6-dihydro-pyridine-3-carboxylic acid [3-(4-fluoro-phenyl)-3-(4-methanesulfonyl-phenyl)-propyl]-amide

### Step A

### 6-Hydroxy-nicotinic acid methyl ester

To the solution of 6-hydroxy nicotinic acid (1.070 g, 7.69 mmol) in benzene (45 mL) and methanol (15 mL) is added TMS diazomethane (5.00 mL, 10.00 mmol) dropwise over 5 minutes. The reaction is stirred at room temperature for 4 hours. The reaction is evaporated in vacuo to give the desired product (1 g, 84.9%). ¹H NMR δ: 8.20 (m, 1H), 8.11 (m, 1H), 7.20 (s, 1H), 6.61 (m, 1H), 3.79 (s, 3H) ppm.

### Step B

### 1-Ethyl-6-hydroxy-nicotinic acid methyl ester

To the solution of the step A product (200 mg, 1.306 mmol) in dichloromethane:acetonitrile (1:1) (10.0 mL), is added PS-TBD (3.20 g, 3.918 mmol) followed by the addition of iodoethane (0.210 mL, 2.658 mmol) and stirred overnight. The solution is then filtered and the resin is washed with dichloromethane and acetonitrile. The resulting solution is condensed in vacuo to give the desired product (0.216 g, 91.3%). LCMS: 182.26 (M+H⁺).

### Step C

### 1-Ethyl-6-hydroxy-nicotinic acid

To lithium hydroxide (0.790 g, 33.00 mmol) and the product from Step B (scaled up; 2.00 g, 11.04 mmol), is added dioxane/water (1:1) (46 mL). The mixture is stirred for 2 hours and dioxane is removed in vacuo. The aqueous layer is washed three times with ethyl acetate, acidified, and the product is extracted with ethyl acetate. The resulting organic layer is condensed in vacuo to give the desired product (1.83 g, 99.2%). LCMS: 168.37 (M+H⁺).

### Step D

### (Z)-3-(4-Fluoro-phenyl)-3-(4-methanesulfonyl-phenyl)-acrylonitrile

A 100 mL flame dried round bottom flask dried under a nitrogen atmosphere is charged with 4-(methanesulphonyl)benzeneboronic acid (1.81 g; 9.1 mmol), 3-chloro-3-(4-fluororphenyl)acrylonitrile (1.52 g; 8.4 mmol), [(C₄H₉)₃PH]⁺BF₄⁻ (0.120 g, 0.42 mmol), Pd₂(dba)₃ (0.191 g, 0.21 mmol). The flask is then placed under vacuum for 5 minutes and backfilled with nitrogen (repeated 3 times). Potassium fluoride (1.490 g, 25.4 mmol) is added to the flask and the flask sealed with a septa, purged with nitrogen and tetrahydrofuran (17.5 mL) is introduced via a syringe. The reaction is placed into an oil bath at 45° C. for 16 hours. The reaction mixture is then filtered though a pad of silica gel, and the silica gel washed with copious amounts of tetrahydrofuran and diethyl ether. The combined organic phase is evaporated in vacuo to give a yellow solid that was recrystallized from hexanes/ethyl acetate to give the desired product (2.08 g, 82% yield). LCMS: 302.06 (M+H⁺).

### Step E

### 3-(4-Fluoro-phenyl)-3-(4-methanesulfonyl-phenyl)-propylamine

A Parr bomb is charged with the product from Step D (150 mg, 0.498 mmol) and 250 mg of 10% pd on carbon. The solids are suspended in ethanol (30 mL) and acetic acid (3 mL) and pressurized to 40 psi in a hydrogentor apparatus. The reaction is run at 40 psi overnight and the product is filtered through a pad of celite. The celite is washed with copious amounts of ethanol and the combined washings evaporated to give an oil which is purified by flash chromatography using methanol/dichloromethan) to give the title compound (0.070 g, 45.7%). LCMS: 308.12 (M+H⁺).

### Step F

To a solution of the product of Example 20 Step C (0.135 g, 0.808 mmol) in dichloromethane is added thionyl chloride, (1.8 mL) and the mixture is stirred for 2 hours. Excess thionyl chloride is removed in vacuo and the oil is taken up in dichloromethane and cooled to 0° C. Triethylamine (0.240 mL) and the product of example 20 Step E (0.170 g, 0.553 mmol) are added to the mixture and allowed to run for 2 hours. The reaction is poured into water and extracted with ether. The ether layers are washed with brine and bicarbonate solution, dried, filtered and evaporated in vacuo. The resulting oil purified by flash chromatography using methanol in dichloromethane (5:95) to give the desired product (0.144 mg, 39.1%) LCMS: 457.535 (M+H⁺).

### EXAMPLE 21

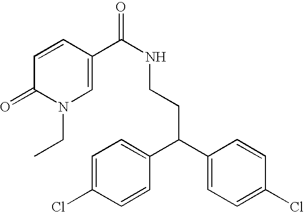

### 1-Cyano-6-oxo-1,6-dihydro-pyridine-3-carboxylic acid [3,3-bis-(4-chloro-phenyl)-propyl]-amide

### Step A

### 3,3-Bis-(4-chloro-phenyl)-2-cyano-acrylic acid ethyl ester

To a solution of (Z)-3-(4-Chloro-phenyl)-2-cyano-acrylic acid ethyl ester (36.00 g, 152.8 mmol) in anhydrous toluene (550 mL) is slowly added 4-chlorophenyl magnesium bromide (1 M in diethyl ether) 199 mL, 198.6 mmol), and the reaction mixture is brought to reflux for 1 h under vigorous stirring, after which the solution is poured into a mixture of ice (400 g) and HCl (4 N) (55 mL). The aqueous layer is separated and washed with ethyl acetate (1×200 mL). The combined organic phase is washed with sodium bicarbonate (1×200 mL), brine, and dried over sodium sulfate. The resulting solution is dried in vacuo to give the desired product (56.00 g, 105.7%).

### Step B

### 2-carbamoyl-3,3-bis-(4-chloro-phenyl)-acrylic acid

To the product from Step A (56.00 g, 150 mmol) is added sodium hydroxide (25.00 g, 600 mmol) in water (500 mL) and the mixture is stirred under reflux for 2 h, cooled to room temperature and washed with MTBE (2×200 mL), acidified with HCl (80 mL), and washed with ethyl acetate (3×200 mL). The ethyl acetate layer is dried over sodium sulfate and evaporated in vacuo to give the product along with two other impurities.

### Step C

### 3,3-Bis-(4-chloro-phenyl)-propionamide and 3,3-bis-(4-chlorophenyl)-propionitrile

To the product from Step B (21 g, 62.09 mmol) in DMSO (200 mL), is added lithium chloride (5.27 g, 124.18 mmol) and the mixture is heated to 130° C. for 2 h and cooled to room temperature. Water (250 mL) and ethyl acetate (100 mL) are added to the mixture and the reaction mixture is shaken vigorously. The organic phase is separated the aqueous phase is extracted with ethyl acetate (100 mL). The combined organic phase, is washed with water (2×100 mL), saturated sodium bicarbonate (100 mL), brine (100 mL), dried over sodium sulfate, filtered and evaporated in vacuo to give the desired products (2:1). LCMS:

### Step D

### 3,3-Bis-(4-chloro-phenyl)-propylamine

To the product from Step C (15.95 g, 54.2 mmol) in tetrahydrofuran (150 mL), is slowly added lithium aluminium hydride (1M in tetrahydrofuran) (110 mL, 110 mmol) at −78° C., the mixture is stirred for 8 h, allowed to come to room temperature, stirred overnight and then cooled down to 0° C. Water (4 mL) is added to the mixture followed by addition of sodium sulfate decahydrate. The resulting salts are filtered off and washed with tetrahydrofuran (5×50 mL). The organic phase is evaporated in vacuo to provide the product as an oil (16 g). This oil is dissolved in MTBE (250 mL), washed with HCl (1×200 mL), brine (1×200 mL) and the solvents are removed in vacuo to provide a foamy yellow solid which is triturated with warm ethyl acetate and warm to give to give a colorless solid. A second crop is obtained from the mother liquor by evaporation in vacuo, re-dissolving in ethyl acetate (20 mL) and precipitation with heptane (250 mL). The combined precipitates are triturated once more from heptane/ethyl acetate (5:1) and filtered to provide colorless powder. The powder is dissolved in heptane/acetone (5:2) (700 mL), heated, cooled and filtered to provide a yellow solid (9.20 g). The solid is boiled in chloroform (100 mL), chilled to −10° C., and filtered to provide a white solid. The filtrate is concentrated, triturated in chloroform and filtered to provide colorless solid which is combined with the other solid (7.89 g, 51.8%). LCMS: 280.74 (M+H⁺).

### Step E

### 1-Cyano-6-oxo-1,6-dihydro-pyridine-3-carboxylic acid [3,3-bis-(4-chlorophenyl)-propyl]-amide

The compound is prepared starting from the product of Example 20 Step C (0.135 g, 0.808 mmol), the product of Example 21, Step D, (0.250 g, 0.790 mmol), EDC (0.303 g, 1.589 mmol), HOBt (0.220 g, 1.628 mmol) and diisopropylethylamine (0.500 mL, 3.869 mmol), and purified by flash chromatography using methanol/dichloromethane as the eluent to give the desired product (0.196 g, 56.5%). LCMS: 430.345 (M+H⁺).

### EXAMPLE 22

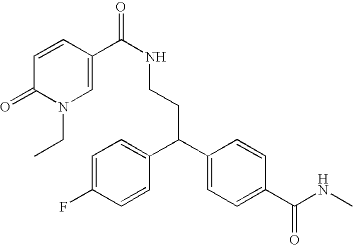

### 1-Ethyl-6-oxo-1,6-dihydro-pyridine-3-carboxylic acid [3-(4-fluoro-phenyl)-3-(4-methylcarbamoyl-phenyl)-propyl]-amide

### Step A

### 6-Hydroxy-nicotinic acid methyl ester

To the solution of 6-hydroxy nicotinic acid (1.070 g, 7.692 mmol) in 54 mL benzene and 15 mL methanol is added TMS diazomethane (5.00 mL, 10.00 mmol) dropwise over 5 minutes. The reaction is stirred at room temperature for 4 h. The reaction is concentrated in vacuo to give the desired product (1.00 g, 84.9%). ¹H NMR δ: 8.20 (m, 1H), 8.11 (m, 1H), 7.20 (s, 1H), 6.61 (m, 1H), 3.79 (s, 3H) ppm.

### Step B

### 1-Ethyl-6-hydroxy-nicotinic acid methyl ester

To the solution of the Step A product (0.200 g, 1.306 mmol) in dichloromethane/acetonitrile

(1:1) (10 mL), is added PS-TBD (3.20 g, 3.918 mmol) followed by the addition of iodoethane (0.210 mL, 2.658 mmol) and stirred overnight. The solution is then filtered and the resin is washed with dichloromethane and acetonitrile. The resulting solution is condensed in vacuo to give the desired product (0.216 g, 91.3%). LCMS: 182.26 (M+H⁺).

### Step C

### 1-Ethyl-6-hydroxy-nicotinic acid

To lithium hydroxide (0.790 g, 33.000 mmol) and the product from Step B (scaled up; 2.00 g, 11.04 mmol) is added dioxane/water (1:1) (46 mL). The mixture is stirred for 2 h and dioxane is removed in vacuo. The aqueous phase is extracted with ethyl acetate, acidified, and the product is extracted with ethyl acetate. The resulting organic phase is condensed in vacuo to give the desired product (1.830 g, 99.2%). LCMS: 168.37 (M+H⁺).

### Step D

### (Z)-3-(4-Fluoro-phenyl)-3-(4-methanesulfonyl-phenyl)acrylonitrile

An oven dried round bottom flask is charged with 4-(N-methylcarboxamide)benzeneboronic acid (0.43 g, 2.43 mmol), 3-chloro-3-(4-fluororphenyl)acrylonitrile (0.4 g, 2.20 mmol), [(C₄H₉)₃PH]⁺BF₄⁻ (0.065 g, 0.22 mmol), Pd₂(dba)₃ (0.1 g, 0.11 mmol). The flask is sealed with a Teflon lined septa cap and then purged with nitrogen for 3 minutes. The cap is removed and then KF is added quickly in one portion. The reaction cap is replaced and the vial flushed with nitrogen for 3 min. Dry tetrahydrofuran (3.6 mL) is introduced into the vial via syringe, and the reaction vessel sealed with Teflon tape. The vial is then immersed in an oil bath with the temperature set to 45° C. and then allowed to react overnight. After 16 h the vial is removed form the oil bath and allowed to come to room temperature. The mixture is filtered trough a plug of silica gel, and the silica gel washed with copious amounts of tetrahydrofuran and diethyl ether. The combined washings were evaporated in vacuo to give the desired compound (0.590 g, 86%). LCMS: 281.11 (M+H⁺).

### Step E

### 4-[3-Amino-1-(4-fluoro-phenyl)-propyl]-N-methyl-benzamide

A parr bomb apparatus is charged with the product from Step D (0.266 g, 0.949 mmol) and Palladium on carbon (0.25 g, 10% v/v?). The solids are suspended in ethanol (30 mL) and acetic acid (3 mL) and pressurized to 40 psi in a hydrogentor apparatus. The reaction is run at 40 psi overnight and the product is filtered through a pad of celite. The celite is washed with copious amounts of ethanol and the combined washings evaporated in vacuo to give an oil which is purified by flash chromatography using methanol in dichloromethane as the eluent to give the title compound (40.0 mg, 14.7%). LCMS: 287.12 (M+H⁺).

### Step F

### 1-Ethyl-6-oxo-1,6-dihydro-pyridine-3-carboxylic acid [3-(4-fluoro-phenyl)-3-(4-methylcarbamoyl-phenyl)-propyl]-amide

The compound is prepared and purified is purified by flash chromatography using methanol/dichloromethane (5:95) as the eluent using the procedure from Example 1 Step A, starting with the product of Example 20 Step C (0.129 g, 0.774 mmol), the product of Example 22, Step E, (0.250 g, 0.774 mmol), EDC (0.293 g, 1.540 mmol), HOBt (0.208 g, 1.54 mmol) and diisopropylethylamine (0.512 mL, 3.09 mmol), to give the desired product (0.196 g, 56.5%). LCMS: 435.98 (M+H⁺).

### Methods of Use

In accordance with the invention, there are provided methods of using the compounds as described herein and their pharmaceutically acceptable derivatives. The compounds used in the invention prevent the degradation of sEH substrates that have beneficial effects or prevent the formation of metabolites that have adverse effects. The inhibition of sEH is an attractive means for preventing and treating a variety of cardiovascular diseases or conditions e.g., endothelial dysfunction. Thus, the methods of the invention are useful for the treatment of such conditions. These encompass diseases including, but not limited to, type 1 and type 2 diabetes, insulin resistance syndrome, hypertension, atherosclerosis, coronary artery disease, angina, ischemia, ischemic stroke, Raynaud's disease and renal disease.

For therapeutic use, the compounds may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds described herein may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the above-described compounds include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

The terms “treating” or “treatment” mean the treatment of a disease-state in a patient, and include:
(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

### In Vitro Assay for Inhibition of hsEH

This high throughput screen identifies compounds that inhibit the interaction of human soluble epoxide hydrolase (sEH) with a tetramethyl rhodamine (TAMRA)-labeled probe. The UHTS employs the Zymark Allegro modular robotic system to dispense reagents, buffers, and test compounds into either 96-well or 384-well black microtiter plates (from Costar). The assay buffer is: 20 mM TES, 200 mM NaCl, 0.05% w/v CHAPS, 1 mM TCEP, pH=7.0. Test compounds dissolved in neat DMSO at 5 mg/mL are diluted to 0.5 mg/mL in neat DMSO. The 0.5 mg/mL solutions are further diluted to 30 μg/mL in assay buffer containing DMSO such that the final concentration of DMSO is 30%. For 384-well format, a mixture of 10.35 nM human sEH and 2.59 nM probe is prepared in assay buffer and 60 μL is added to each well for a final sEH concentration of 10 nM and a final probe concentration of 2.5 nM. 2.1 μL of diluted test compound is then added to each well, where the final assay concentration will be 1 μg/mL test compound and 1% DMSO. The final volume in each well is 62.1 μL. Positive controls are reaction mixtures containing no test compound; negative controls (blanks) are reaction mixtures containing 3 μM BI00611349XX. For 96-well format, the final concentration of all reaction components remains the same. 135 μL sEH/probe mixture is added to wells containing 15 μL test compound so that the final well volume is 150 mL. After incubating the reaction for 30 minutes at room temperature, the plates are read for fluorescence polarization in the LJL Analyst set to 530 nm excitation, 580 nm emission, using the Rh 561 dichroic mirror.

### In Vitro Assay for Inhibition of msEH

This screen identifies compounds that inhibit the interaction of rat soluble epoxide hydrolase (sEH) with a tetramethyl rhodamine (TAMRA)-labeled probe. The assay employs a Multimek, a Multidrop, and manual multi-channel pipettors to dispense reagents, buffers, and test compounds into 96-well black microtiter plates (Costar 3792). The assay buffer is: 20 mM TES, 200 mM NaCl, 0.05% w/v CHAPS, 1 mM TCEP, pH=7.0. Test compounds dissolved in neat DMSO at 10 mM are diluted to 1.5 mM in neat DMSO. The 1.5 mM solutions are serially diluted using 3-fold dilutions in neat DMSO in polypropylene plates. Assay buffer is added to the wells such that the compounds are diluted 10-fold and the DMSO concentration is 10%. A mixture of 11.1 nM rat sEH and 2.78 nM probe is prepared in assay buffer. 15 uL of diluted test compound is added to each well, where the final maximum assay concentration will be 3 uM test compound and 1% DMSO. 135 uL of sEH/probe mixture is added to each well for a final sEH concentration of 10 nM and a final probe concentration of 2.5 nM. The final volume in each well is 150 uL. Positive controls are reaction mixtures containing no test compound; negative controls (blanks) are reaction mixtures containing 3 uM B100611349XX. After incubating the reaction for 30 minutes at room temperature, the plates are read for fluorescence polarization in the LJL Analyst set to 530 nm excitation, 580 nm emission, using the Rh 561 dichroic mirror.

1. A compound of the formula (I): 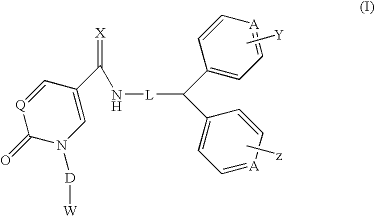
each A is independently nitrogen or C—H such that each of the ring of which A is a member may be pyridinyl or phenyl, said pyridinyl or phenyl are optionally substituted by Y or Z;
Y and Z on their respective rings are in the meta or para position, and are independently F, Cl, Br, CN, OR, R, —S(O)₂R, —C(O)NRR or —S(O)₂NRR, wherein R is independently hydrogen or C₁₋₅ alkyl unsubstituted or substituted with hydroxy, amino, C₁₋₄ alkoxy, C₁₋₄ alkylamino, C₁₋₄ alkylthio, or one to three fluorine atoms;
L is an ethylene linker optionally substituted with hydroxy, amino, C₁₋₄ alkoxy C₁₋₄ alkylamino, C₁₋₄ alkylthio, or one to three fluorine atoms;
X is O or S;
Q is N or CH;
D is a bond, or a methylene or ethylene linker, wherein a —CH₂— group of said linker may be replaced by —C(O)—;
W is hydrogen, C1-5 alkyl, cyano, carbocycle, heterocylyl, aryloxy, C1-4 alkyloxy, OH or heteroaryl; each being unsubstituted or substituted with hydroxy, amino, halogen, cyano, carboxy, carboxamido, C1-4 alkyl, C1-4 alkylthio, C1-4 alkyloxy, C1-4 alkylamino, C1-4 dialkylamino, C3-6 cycloalkylamino, di(C3-6 cycloalkyl)amino, C1-4 alkylsulfonyl, C1-4 alkyloxycarbonyl or C1-4 alkylamidocarbonyl;
wherein if D is a bond then W is cyano,
or the pharmaceutically acceptable salts thereof.

2. The compound according to claim 1 and wherein:
Q is CH;
X is O;
Y and Z, if present, on their respective rings are in the meta or para position, and are independently F, Cl, —S(O)₂R or —C(O)NRR wherein R is independently hydrogen or C1-5 alkyl;
W is hydrogen, cyano, C3-6 cycloalkyl, aryl, phenoxy, C1-4 alkyloxy, OH, C1-5 alkyl, heterocylyl chosen from piperidinyl, morpholinyl, thiomorpholinyl, piperazinyl, pyrrolidinyl and dioxolanyl or heteroaryl chosen from pyrazolyl, pyrrolyl, imidazolyl, furanyl, pyranyl, thienyl, thiazolyl, oxazolyl, pyridinyl, pyrimidinyl, pyrazinyl, pyridazinyl and 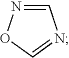
each being unsubstituted or substituted with hydroxy, amino, halogen, cyano, carboxy, carboxamido, C1-4 alkyl, C1-4 alkylthio, C1-4 alkyloxy, C1-4 alkylamino, C1-4 dialkylamino, C3-6 cycloalkylamino, di(C3-6 cycloalkyl)amino, C1-4 alkylsulfonyl, C1-4 alkyloxycarbonyl or C1-4 alkylamidocarbonyl.

3. The compound according to claim 2 and wherein:
L is an ethylene linker;
W is hydrogen, cyano, piperidinyl, morpholinyl, phenyl, phenoxy, C1-4 alkyloxy, OH, C1-5 alkyl, pyridinyl or 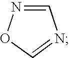
each being unsubstituted or substituted with hydroxy or C₁₋₄ alkyloxy.

4. A compound of the formula (II): 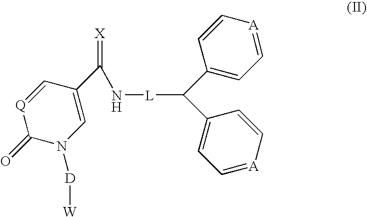
wherein for the Formula (II), the component 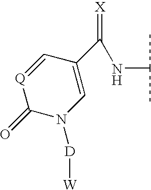
is chosen from A1-A14 in the table I below; in combination with any component 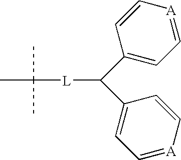
chosen from B1-B11 in the table below:

| | 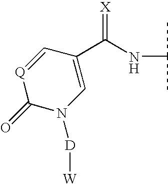 |
|---|---|
| A1 | 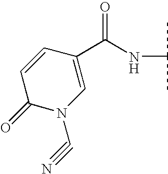 |
| A2 | 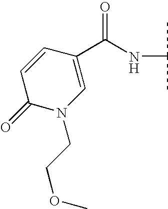 |
| A3 | 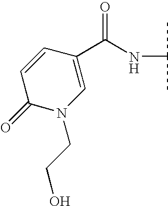 |
| A4 | 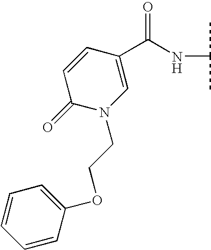 |
| A5 | 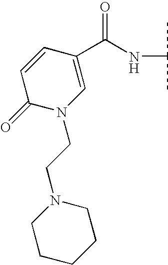 |
| A6 | 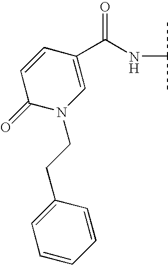 |
| A7 | 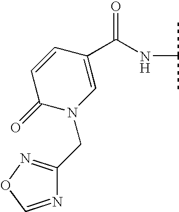 |
| A8 | 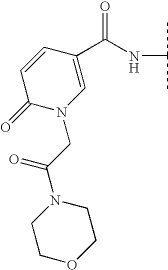 |
| A9 | 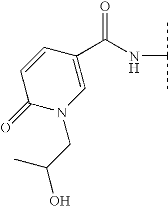 |
| A10 | 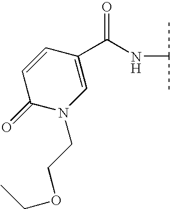 |
| A11 | 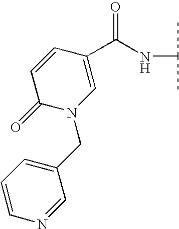 |
| A12 | 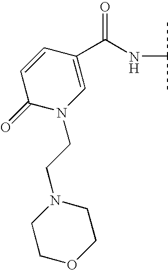 |
| A13 | 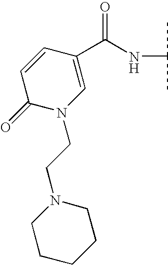 |
| A14 | 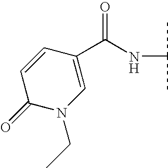 |
| | 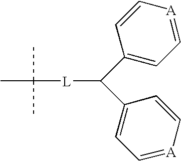 |
| B1 | 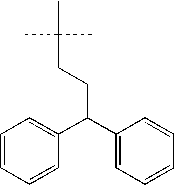 |
| B2 | 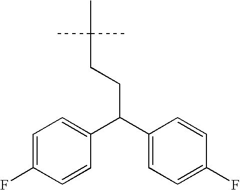 |
| B3 | 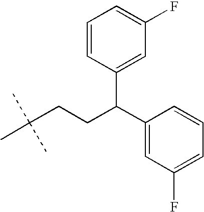 |
| B4 | 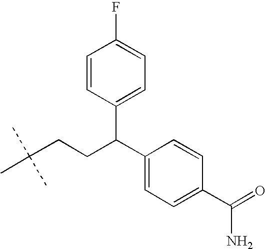 |
| B5 | 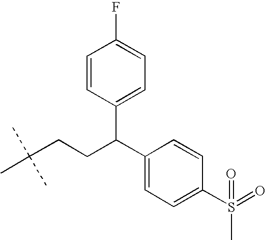 |
| B6 | 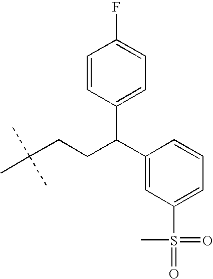 |
| B7 | 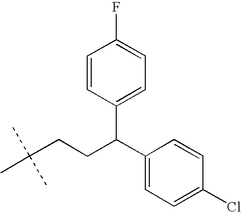 |
| B8 | 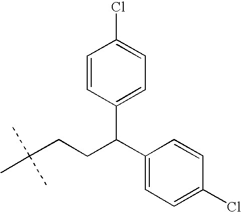 |
| B9 | 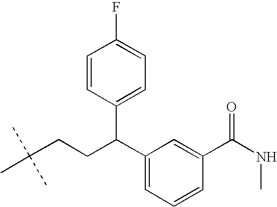 |
| B10 | 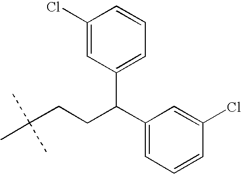 |
| B11 | 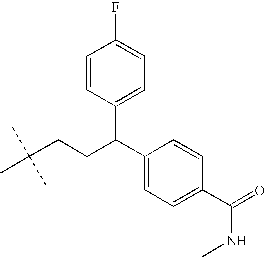 |

or the pharmaceutically acceptable salts thereof.

5. A compound chosen from 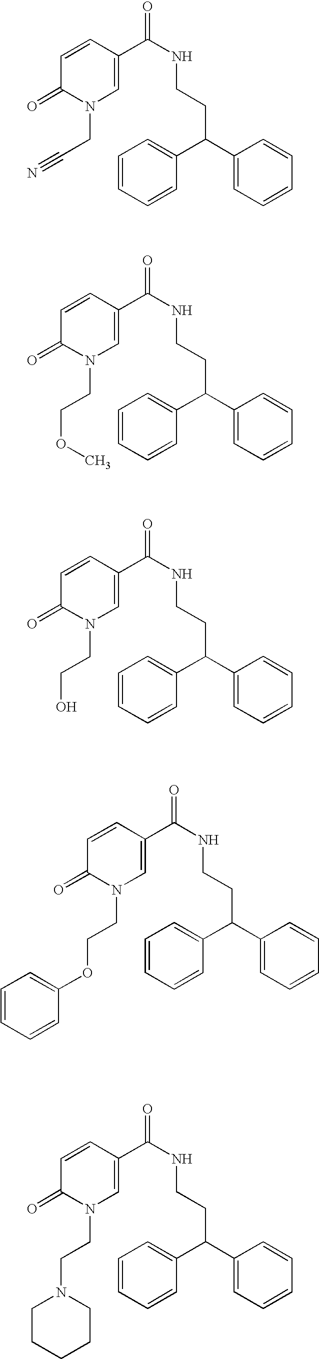 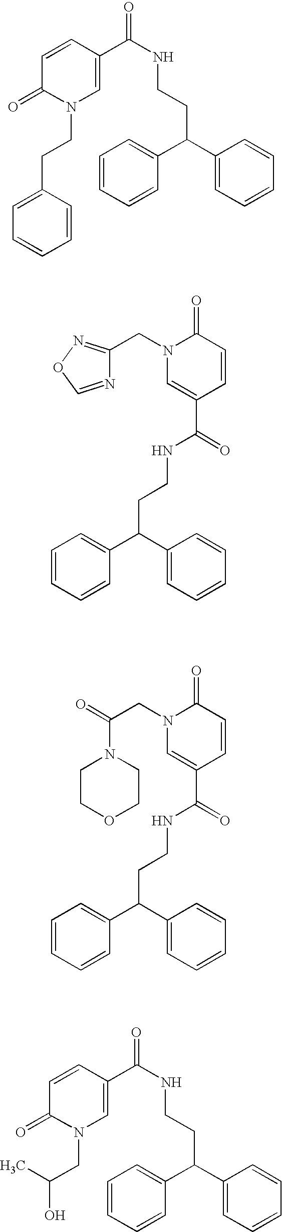 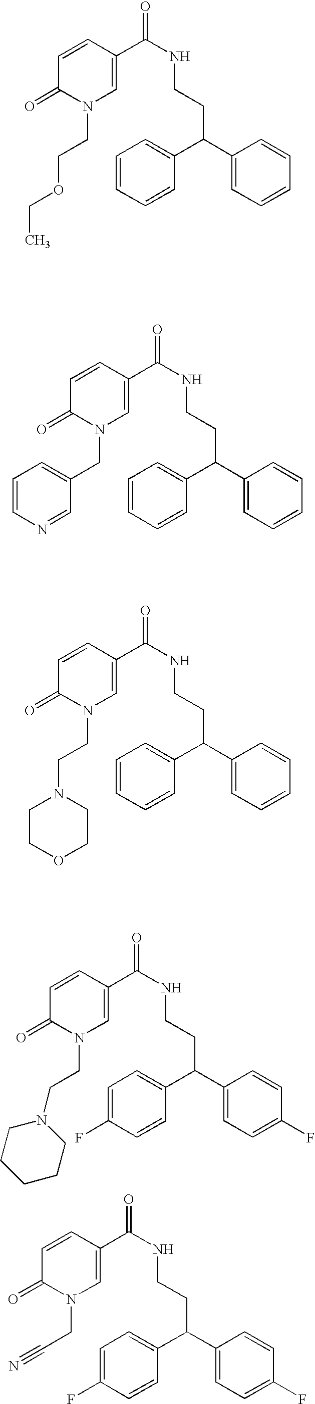 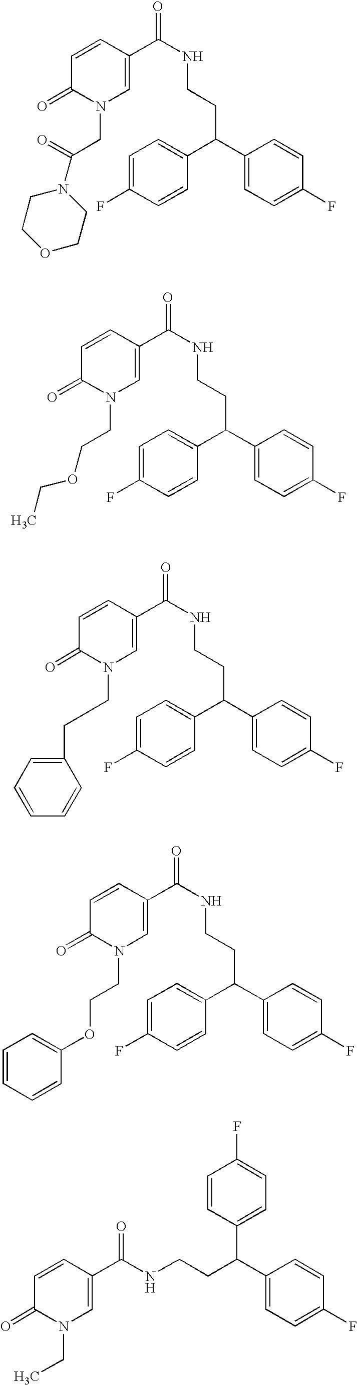 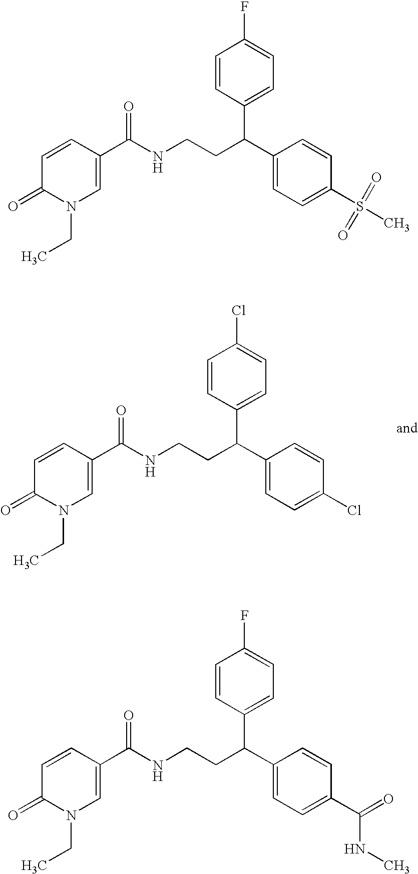
or the pharmaceutically acceptable salts thereof.

6. A method of treating a disease chosen from type 1 and type 2 diabetes, insulin resistance syndrome, hypertension, atherosclerosis, coronary artery disease, angina, ischemia, ischemic stroke, Raynaud's disease and renal disease comprising administering to a patient a therapeutically effective amount of a compound according to any one of claims 1-5.
